(19) United States
(12) Patent Application Publication
(10) Pub. No.: US 2021/0186622 A1
(43) Pub. Date: Jun. 24, 2021

(54) DEVICE, SYSTEM AND/OR METHOD FOR POSITION TRACKING

(71) Applicants: Desmond Hirson, Toronto (CA); Claudio Irrgang, Toronto (CA); VENTRIPOINT DIAGNOSTICS LTD., Toronto (CA)

(72) Inventors: Desmond Hirson, Toronto (CA); Claudio Irrgang, Toronto (CA)

(21) Appl. No.: 16/757,755

(22) PCT Filed: Oct. 18, 2018
(86) PCT No.: PCT/CA2018/051309

§ 371 Date: Apr. 20, 2020

(30) Foreign Application Priority Data
Oct. 19, 2017 (CA) PCT/CA17/051248

(51) Int. Cl. A61B 34/20; A61B 90/00; G01S 15/89; G01B 7/004

(52) U.S. Cl. CPC A61B 34/20; A61B 90/37; A61B 2017/00221; G01B 7/004; G01S 15/899

(57) ABSTRACT

Disclosed is a device, system and method for use with sensors including an accelerator, a gyroscope and magnetometers to determine position and orientation of a probe. The probe may be attached to, or incorporate other instruments such as ultrasound transducer and surgical instruments. Each of the sensors generate sensor data, including calibration data. The system may also have a second position sensor in proximity to the probe. The system also has a processing engine receiving the sensor data for calculating the position and orientation of the probe relative to the second position sensor.

### FIELD OF THE INVENTION

The present invention relates generally to a handheld probe for position tracking in three-dimensional space including orientation in three axes. In particular, the present invention relates to the use of a reference magnet and MEMS sensors to provide high resolution x, y and z coordinates and orientation of an instrument.

### BACKGROUND OF THE INVENTION

In the prior art, different solutions may have been developed for attempting to determine the absolute position information for location tracking. One such system may be the Global Positioning System (“GPS”), which does not work for indoor positioning system applications. GPS relies on a device having unfettered communication access to satellites, which may not be possible for indoors (e.g., in an office). GPS typically requires a minimum of three satellites in view of the device in order to triangulate a position. When this is not possible, the GPS “drops out”. Also, by design, GPS may have limits imposed on its accuracy and is typically about +/−10 m in the x and y-axes and +/−20 m in the z-axis.

In the prior art, internal navigation systems (“INS”) were developed to address the problems encountered with devices attempting to communicate with satellites. In some systems, INS takes over when the GPS drops out or is associated with large errors. In some applications, such as onboard systems for aircraft navigation, the resolution with INS is sufficient. For other applications, such as human-based tracking or applications where millimeter level accuracy is required, typically INS may not be effective. In addition, many INS are typically very large and expensive.

In other applications in the prior art, systems may use electromagnetic sensing for position determination but are typically, bulky, expensive and problematic for use with a patient in a clinical setting. In addition, systems may use line-of-sight and stereotactic cameras for position tracking.

As a result, there may be a need for, or it may be desirable to provide a device, system, method and/or cooperating environment that overcomes one or more of the limitations associated with the prior art. It may be advantageous to provide a device, system and/or method for position tracking with improved accuracy, a compact size and/or a low cost.

### SUMMARY OF THE INVENTION

The present disclosure provides a device, system and/or method for determining the position and orientation of a probe. The probe or first position sensor includes sensors such as an accelerometer, a gyroscope and magnetometers. The probe may be attached to, or incorporate, instruments such as an ultrasound transducer and/or surgical instruments. Each of the sensors generate sensor data. The system may have a second position sensor (such as a reference magnet) in proximity to the probe. The system also has a processor receiving the sensor data for calculating a position and orientation of the probe relative to the second position sensor.

According to the invention, there is disclosed a system for use by a user with a patient. The system includes a first position sensor having an accelerometer adapted to receive accelerometer data associated with the first position sensor, a gyroscope adapted to receive gyroscope data associated with the first position sensor, and a magnetometer adapted to receive magnetometer data associated with the first position sensor. The system also includes one or more processors that are operative to electronically receive the accelerometer data, the gyroscope data and the magnetometer data and analyze the accelerometer data, the gyroscope data and the magnetometer data using an analysis algorithm to automatically generate position and orientation data associated with the first position sensor. One or more databases are also included in the system to electronically store the accelerometer data, the gyroscope data, the magnetometer data and the position and orientation data. Thus, according in the invention, the system is operative to facilitate the determination of the position and orientation of the first position sensor.

According to an aspect of one preferred embodiment of the invention, the position probe system may preferably, but need not necessarily, include a second position sensor associated with the patient to facilitate the determination of the position and orientation of the first position sensor relative to the patient.

According to an aspect of one preferred embodiment of the invention, the second position sensor may preferably, but need not necessarily, be a reference magnet.

According to an aspect of one preferred embodiment of the invention, the second position sensor may preferably, but need not necessarily, be secured to the patient to compensate for movement of the patient during the determination of the position and orientation of the first position sensor.

According to an aspect of one preferred embodiment of the invention, the accelerometer data, the gyroscope data, and the magnetometer data may preferably, but need not necessarily, comprise an x-axis, a y-axis, and/or a z-axis.

According to an aspect of one preferred embodiment of the invention, the accelerometer data, the gyroscope data, and the magnetometer data is time-stamped.

According to an aspect of one preferred embodiment of the invention, the first position sensor may preferably, but need not necessarily, be associated with an ultrasound transducer or a surgical instrument.

According to an aspect of one preferred embodiment of the invention, the first position sensor may preferably, but need not necessarily, be removably mounted to the ultrasound transducer or the surgical instrument.

According to an aspect of one preferred embodiment of the invention, the first position sensor may preferably, but need not necessarily, be integral with the ultrasound transducer or the surgical instrument.

According to an aspect of one preferred embodiment of the invention, the one or more databases may preferably, but need not necessarily, include a device database, local to the first position sensor, to electronically store the accelerometer data, the gyroscope data, the magnetometer data and the position and orientation data.

According to an aspect of one preferred embodiment of the invention, the first position sensor may preferably, but need not necessarily, further include interface features to facilitate interaction by the user with the sensor.

According to an aspect of one preferred embodiment of the invention, the first position sensor may preferably, but need not necessarily, further include a light pipe to illuminate the sensor.

According to the invention, there is also disclosed a method for determining the position and orientation of a first position sensor for use with a patient by a user. The method includes steps (a), (b), and (c). Step (a) involves operating a first position sensor that includes an accelerometer adapted to receive accelerometer data associated with the first position sensor, a gyroscope adapted to receive gyroscope data associated with the first position sensor, and a magnetometer adapted to receive magnetometer data associated with the first position sensor. Step (b) involves operating one or more processors to electronically receive the accelerometer data, the gyroscope data and the magnetometer data and execute an analysis algorithm to analyze the accelerometer data, the gyroscope data and the magnetometer data to automatically generate position and orientation data associated with the first position sensor. Step (c) involves electronically storing the accelerometer data, the gyroscope data and the magnetometer data and the position and orientation data in one or more databases. Thus, according to the invention, the method operatively facilitates the determination of the position and orientation of the first position sensor.

According to an aspect of one preferred embodiment of the invention, the method preferably, but need not necessarily, further includes a step of associating a second position sensor with the patient to facilitate the determination of the position and orientation of the first position sensor relative to the patient.

According to an aspect of one preferred embodiment of the invention, the method preferably, but need not necessarily, further includes a step of associating the second position sensor with the patient whereby the second position sensor is a reference magnet.

According to an aspect of one preferred embodiment of the invention, the method preferably, but need not necessarily, further includes a step of securing the second position sensor to the patient to compensate for movement of the patient during the determination of the position and orientation of the first position sensor.

According to an aspect of one preferred embodiment of the invention, in step (a), the accelerometer data, the gyroscope data, and the magnetometer data may preferably, but need not necessarily, include an x-axis, a y-axis and/or a z-axis.

According to an aspect of one preferred embodiment of the invention, in step (a), the accelerometer data, the gyroscope data, and the magnetometer data may preferably, but need not necessarily, be associated with a time-stamp.

According to an aspect of one preferred embodiment of the invention, in step (a), the first position sensor may preferably, but need not necessarily, be associated with an ultrasound transducer or a surgical instrument.

According to an aspect of one preferred embodiment of the invention, the method preferably, but need not necessarily, further includes a step of removably mounting the first position sensor to the ultrasound transducer or the surgical instrument.

According to an aspect of one preferred embodiment of the invention, the method preferably, but need not necessarily, further includes a step of calibration for the accelerometer, the gyroscope and the magnetometer to generate calibration data.

According to an aspect of one preferred embodiment of the invention, in step (c), the one or more databases may preferably, but need not necessarily, include a device database local to the first position sensor to store the accelerometer data, the gyroscope data, the magnetometer data, the position and orientation data, and the calibration data.

According to the invention, there is disclosed a first position sensor, operated by a user. The first position sensor includes an accelerometer adapted to collect accelerometer data associated with the first position sensor, a gyroscope adapted to collect gyroscope data associated with the first position sensor, and a first magnetometer adapted to collect magnetometer data associated with the first position sensor, and one or more processors. The one or more processors are operative to receive the accelerometer data, the gyroscope data and the magnetometer data and automatically apply an analysis algorithm to generate position and orientation data associated with the first position sensor, and together with the accelerometer data, the gyroscope data and the magnetometer data is electronically stored in one or more databases. Thus, according to the invention, the first position sensor is operative to facilitate determination of the position and orientation of the first position sensor.

According to an aspect of one preferred embodiment of the invention, the first position sensor may preferably, but need not necessarily, further include a second magnetometer adapted to collect the magnetometer data associated with the first position sensor.

According to an aspect of one preferred embodiment of the invention, the accelerometer, the gyroscope, the first magnetometer and the second magnetometer may preferably, but need not necessarily, be tri-axial.

According to an aspect of one preferred embodiment of the invention, the one or more databases may preferably, but need not necessarily, include a device database local to the first position sensor to electronically store the accelerometer data, the gyroscope data and the magnetometer data.

According to an aspect of one preferred embodiment of the invention, the first position sensor may preferably, but need not necessarily, further include one or more interface features to facilitate interaction between the user and the sensor.

According to an aspect of one preferred embodiment of the invention, the one or more interface features include buttons, switches, display screens, interactive screens, and/or indicator lights.

According to an aspect of one preferred embodiment of the invention, the first position sensor may preferably, but need not necessarily, further include a light pipe adapted to illuminate the sensor.

According to an aspect of one preferred embodiment of the invention, the first position sensor processor is preferably, but need not necessarily, in communication with and/or detects a second position sensor associated with a patient to facilitate the determination of the position and orientation of the first position sensor relative to the patient.

According to the invention, there is disclosed a computer readable medium on which is physically stored executable instructions. The executable instructions are such as to, upon execution, determine the position and orientation of a first position sensor operated by a user with a patient. The executable instructions include processor instructions for a device processor and/or a base station processor to automatically and according to the invention: (a) collect and/or electronically communicate accelerometer data from the device processor to the base station processor; (b) collect and/or electronically communicate gyroscope data from the device processor to the base station processor; (c) collect and/or electronically communicate magnetometer data from the device processor to the base station processor; (d) automatically generate position and orientation data associated with the first position sensor using an analysis algorithm; and (e) electronically store the accelerometer data, the gyroscope data, the magnetometer data, and the position and orientation data in a base station database. Thus, according to the invention, the computer readable medium operatively facilitates the determination of the position and orientation of the first position sensor.

Other advantages, features and characteristics of the present invention, as well as methods of operation and functions of the related elements of the device, system and/or method, and the combination of steps, parts and economies of manufacture, will become more apparent upon consideration of the following detailed description and the appended claims with reference to the accompanying drawings, the latter of which are briefly described herein below.

### BRIEF DESCRIPTION OF THE DRAWINGS

The novel features which are believed to be characteristic of the device, system, and/or method according to the present invention, as to their structure, organization, use, and method of operation, together with further objectives and advantages thereof, may be better understood from the following drawings in which presently preferred embodiments of the invention may now be illustrated by way of example. It is expressly understood, however, that the drawings are for the purpose of illustration and description only, and are not intended as a definition of the limits of the invention. In the accompanying drawings:

FIG. 1 is a schematic diagram of a system and device for collecting and/or analyzing sensor data according to one preferred embodiment of the invention;

FIG. 2 is a schematic diagram of components of the system and device of FIG. 1;

FIG. 3 is a schematic diagram of a probe, reference magnet and work area;

FIG. 4 is a schematic view of a probe; and

FIG. 5 is a flowchart of an over-arching method according to a preferred embodiment of the invention.

### DETAILED DESCRIPTION OF THE PREFERRED EMBODIMENTS

The description that follows, and the embodiments described therein, may be provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order to more clearly depict certain embodiments and features of the invention.

The present disclosure may be described herein with reference to system architecture, block diagrams and flowchart illustrations of methods, and computer program products according to various aspects of the present disclosure. It may be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flow diagram illustrations support combinations of means for performing the specified functions, combinations of steps for performing the specified functions, and program instruction means for performing the specified functions. It may also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

The present disclosure may be now described in terms of an exemplary system in which the present disclosure, in various embodiments, would be implemented. This may be for convenience only and may not be intended to limit the application of the present disclosure. It may be apparent to one skilled in the relevant art(s) how to implement the present disclosure in alternative embodiments.

In this disclosure, a number of terms and abbreviations may be used. The following definitions and descriptions of such terms and abbreviations are provided in greater detail.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

In the description and drawings herein, and unless noted otherwise, the terms “vertical”, “lateral” and “horizontal”, are generally references to a Cartesian co-ordinate system in which the vertical direction generally extends in an “up and down” orientation from bottom to top (y-axis) while the lateral direction generally extends in a “left to right” or “side to side” orientation (x-axis). In addition, the horizontal direction extends in a “front to back” orientation and can extend in an orientation that may extend out from or into the page (z-axis).

As used in the specification, there may be defined three axes of rotation with respect to the apparatus. Each axis of this coordinate system is perpendicular to the other two axes. For example, the pitch axis is perpendicular to the yaw axis and the roll axis. A pitch motion or “pitch” is a rotation of the apparatus along the z-axis. A yaw motion or “yaw” is a rotation of the apparatus along the y-axis. A roll motion or “roll” is a rotational movement of the apparatus along the x-axis.

As used herein, a person skilled in the relevant art may generally understand the term “magnetometer” to generally mean an instrument that measures magnetism (i.e., the magnetization of a magnetic material such as a ferromagnet, or the direction, strength, or relative change of a magnetic field at a particular location. Magnetometers may be incorporated in integrated circuits (e.g., a microelectromechnicalsystems or MEMS magnetometer).

As used herein, a person skilled in the relevant art may generally understand the term “accelerometer” to generally mean an instrument that measures the rate of change of velocity (i.e., acceleration) of a body. Single and multi-axis accelerometers may detect magnitude and direction of proper acceleration as a vector quantity and can be used to sense orientation, coordinate acceleration, vibration, shock and falling in a resistive medium. Accelerometers may be incorporated in integrated circuits (e.g., a MEMS accelerometer).

As used herein, a person skilled in the relevant art may generally understand the term “gyroscope” to generally mean an instrument for measuring orientation and angular velocity. Gyroscopes may be incorporated in integrated circuits (e.g., a MEMS gyroscope).

It should also be appreciated that the present invention can be implemented in numerous ways, including as a device, a method or a system. In this specification, these implementations, or any other form that the invention may take, may be referred to as processes. In general, the order of the steps of the disclosed processes may be altered within the scope of the invention.

It may generally be understood by a person skilled in the relevant art that the term “cloud computing” is an information technology model that facilitates ubiquitous access to shared pools of configurable system resources and higher-level services that can be provisioned with minimal management effort, usually over the Internet. Third-party clouds preferably enable organizations to focus on their core businesses instead of allocating resources on computer infrastructure and maintenance.

It may be further generally understood by a person skilled in the relevant art that the term “downloading” refers to receiving datum or data to a local system (e.g., a mobile device) from a remote system (e.g., a client) or to initiate such a datum or data transfer. Examples of a remote systems or clients from which a download might be performed include, but are not limited to, web servers, FTP servers, email servers, or other similar systems. A download can mean either any file that may be offered for downloading or that has been downloaded, or the process of receiving such a file. A person skilled in the relevant art may understand the inverse operation, namely sending of data from a local system (e.g., a mobile device) to a remote system (e.g., a database) may be referred to as “uploading”. The data and/or information used according to the present invention may be updated constantly, hourly, daily, weekly, monthly, yearly, etc. depending on the type of data and/or the level of importance inherent in, and/or assigned to, each type of data. Some of the data may preferably be downloaded from the Internet, by satellite networks or other wired or wireless networks.

Elements of the present invention may be implemented with computer systems which are well known in the art. Generally speaking, computers include a central processor, system memory, and a system bus that couples various system components including the system memory to the central processor. A system bus may be any of several types of bus structures including a memory bus or memory controller, a peripheral bus, and a local bus using any of a variety of bus architectures. The structure of a system memory may be well known to those skilled in the art and may include a basic input/output system (“BIOS”) stored in a read only memory (“ROM”) and one or more program modules such as operating systems, application programs and program data stored in random access memory (“RAM”). Computers may also include a variety of interface units and drives for reading and writing data. A user of the system can interact with the computer using a variety of input devices, all of which are known to a person skilled in the relevant art.

One skilled in the relevant art would appreciate that the device connections mentioned herein are for illustration purposes only and that any number of possible configurations and selection of peripheral devices could be coupled to the computer system.

Computers can operate in a networked environment using logical connections to one or more remote computers or other devices, such as a server, a router, a network personal computer, a peer device or other common network node, a wireless telephone or wireless personal digital assistant. The computer of the present invention may include a network interface that couples the system bus to a local area network (“LAN”). Networking environments are commonplace in offices, enterprise-wide computer networks and home computer systems. A wide area network (“WAN”), such as the Internet, can also be accessed by the computer or mobile device.

It may be appreciated that the type of connections contemplated herein are exemplary and other ways of establishing a communications link between computers may be used in accordance with the present invention, including, for example, mobile devices and networks. The existence of any of various well-known protocols, such as TCP/IP, Frame Relay, Ethernet, FTP, HTTP and the like, may be presumed, and computer can be operated in a client-server configuration to permit a user to retrieve and send data to and from a web-based server. Furthermore, any of various conventional web browsers can be used to display and manipulate data in association with a web-based application.

The operation of the network ready device (i.e., a mobile device) may be controlled by a variety of different program modules, engines, etc. Examples of program modules are routines, algorithms, programs, objects, components, data structures, etc. that perform particular tasks or implement particular abstract data types. It may be understood that the present invention may also be practiced with other computer system configurations, including multiprocessor systems, microprocessor-based or programmable consumer electronics, network PCS, personal computers, minicomputers, mainframe computers, and the like. Furthermore, the invention may also be practiced in distributed computing environments where tasks are performed by remote processing devices that are linked through a communications network. In a distributed computing environment, program modules may be located in both local and remote memory storage devices.

Embodiments of the present invention can be implemented by a software program for processing data through a computer system. It may be understood by a person skilled in the relevant art that the computer system can be a personal computer, mobile device, notebook computer, server computer, mainframe, networked computer (e.g., router), workstation, and the like. In one embodiment, the computer system includes a processor coupled to a bus and memory storage coupled to the bus. The memory storage can be volatile or non-volatile (i.e., transitory or non-transitory) and can include removable storage media. The computer can also include a display, provision for data input and output, etc. as may be understood by a person skilled in the relevant art.

Some portion of the detailed descriptions that follow are presented in terms of procedures, steps, logic block, processing, and other symbolic representations of operations on data bits that can be performed on computer memory. These descriptions and representations are the means used by those skilled in the data processing arts to most effectively convey the substance of their work to others skilled in the art. A procedure, computer executed step, logic block, process, etc. is here, and generally, conceived to be a self-consistent sequence of operations or instructions leading to a desired result. The operations are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following description, it is appreciated that throughout the present invention, references utilizing terms such as “receiving”, “creating”, “providing”, “communicating” or the like refer to the actions and processes of a computer system, or similar electronic computing device, including an embedded system, that manipulates and transfers data represented as physical (electronic) quantities within the computer system's registers and memories into other data similarly represented as physical quantities within the computer system memories or registers or other such information storage, transmission or display devices.

The present invention is contemplated for use in association with one or more cooperating environments, to afford increased functionality and/or advantageous utilities in association with same. The invention, however, is not so limited.

Certain novel features which are believed to be characteristic of a device, system, method for position tracking in three-dimensional space and/or certain features of the device, system, method which are novel in conjunction with the cooperating environment, according to the present invention, as to their organization, use, and/or method of operation, together with further objectives and/or advantages thereof, may be better understood from the accompanying disclosure in which presently preferred embodiments of the invention are disclosed by way of example. It is expressly understood, however, that the accompanying disclosure is for the purpose of illustration and/or description only, and is not intended as a definition of the limits of the invention.

Naturally, in view of the teachings and disclosures herein, persons having ordinary skill in the art may appreciate that alternate designs and/or embodiments of the invention may be possible (e.g., with substitution of one or more steps, algorithms, processes, features, structures, parts, components, modules, utilities, etc. for others, with alternate relations and/or configurations of steps, algorithms, processes, features, structures, parts, components, modules, utilities, etc.).

Although some of the steps, algorithms, processes, features, structures, parts, components, modules, utilities, relations, configurations, etc. according to the invention are not specifically referenced in association with one another, they may be used, and/or adapted for use, in association therewith.

One or more of the disclosed steps, algorithms, processes, features, structures, parts, components, modules, utilities, relations, configurations, and the like may be implemented in and/or by the invention, on their own, and/or without reference, regard or likewise implementation of one or more of the other disclosed steps, algorithms, processes, features, structures, parts, components, modules, utilities, relations, configurations, and the like, in various permutations and combinations, as may be readily apparent to those skilled in the art, without departing from the pith, marrow, and spirit of the disclosed invention.

In certain implementations, instructions may include those for the analysis of sensor data. While computer-readable storage medium may be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and software components, or only in software.

In the present description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

The present disclosure also relates to an apparatus for performing the operations herein. This apparatus may be specially constructed for the required purposes, or it may include a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (“ROMs”), random access memories (“RAMs”), EPROMs, EEPROMs, magnetic or optical cards, or any type of media suitable for storing electronic instructions.

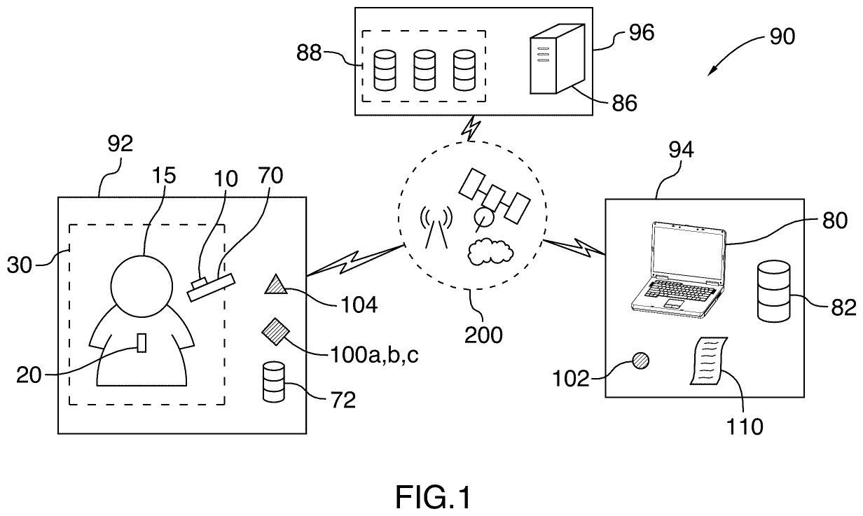

Some parts of the system 90 depicted in FIG. 1 may be provided at a remote location. Preferably, and as best seen in FIG. 1, the system 90 includes a device subsystem 92, a base station subsystem 94, and an accessory subsystem 96.

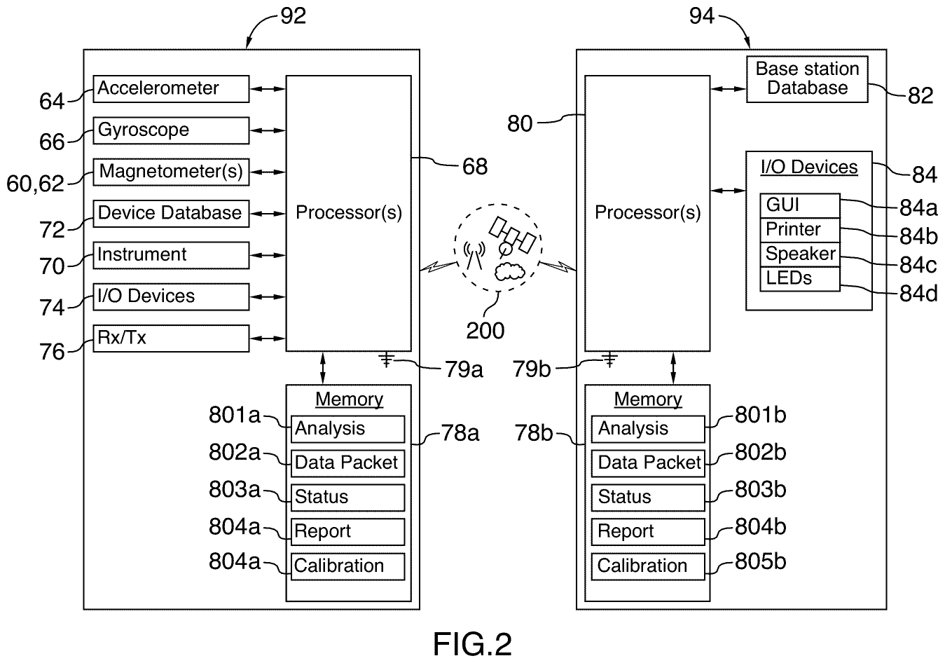

In FIGS. 1 and 2, the system 90 is shown in use with a communication network 200. The communication network 200 may include satellite networks (e.g., GPS), terrestrial wireless networks, and the Internet. The communication of data between the device subsystem 92, the base station subsystem 94 and/or the accessory subsystem 96 may also be achieved via one or more wired means of transmission (e.g., docking the probe 10 in a base of the base station subsystem 94), or other physical means (e.g., a Universal Serial Bus cable and/or flash drive) of transmission. Persons having ordinary skill in the art will appreciate the system 90 includes hardware and software.

FIG. 2 schematically illustrates, among other things, that the device subsystem 92 includes a probe 10 (alternately “first position sensor 10”) for measuring or receiving sensor data 100 and a first magnetometer 60, a second magnetometer 62, an accelerometer 64, a gyroscope 66, a controller or processor 68, an instrument 70, a device database 72, device input/output (or “I/O”) components (e.g., display, auditory, and/or tactile components) 74, a transmitter-receiver 76, and/or a computer readable medium (e.g., an onboard device processor-readable memory) 78a local to the device controller or processor 68. Each of the first magnetometer 60, second magnetometer 62, accelerometer 64, gyroscope 66, controller or device processor 68, instrument 70, device database 72, device input/output components 74, and transmitter-receiver 76 may collectively be referred to as a component(s) 300. The base station subsystem 94 includes a base station processor 80 (which may preferably be provided as a component of a tablet, laptop, computer, smart phone, server or any other device that may be known to a person of skill in the art), a base station database 82, input-output devices (e.g., printer for generating reports, etc.) 84, and/or a computer readable medium (e.g., a processor-readable memory) 78b local to the base station processor 80. The accessory subsystem 96 may include an accessory processor 86 and/or remote databases 88.

As best seen in FIGS. 1, 2 and 4, the probe 10 preferably includes the first magnetometer 60, the second magnetometer 62, the accelerometer 64, the gyroscope 66, the controller 68, the instrument 70, the device database 72, the device input/output components 74, and the transmitter-receiver 76.

Preferably, the probe 10 uses the magnetometers 60, 62, the accelerometer 64, and the gyroscope 66 to automatically receive magnetometer data 100a, accelerometer data 100b and gyroscope data 100c (collectively “sensor data 100”) associated with the position of the probe 10 within a predetermined work area 30. Using the transmitter-receiver 76, the controller 68 may wirelessly communicate via the communication network 200 (for example, by the Bluetooth™ Low Energy proprietary open wireless technology standard which is managed by the Bluetooth Special Interest Group of Kirkland, Wash.) with—or may be wired to communicate with—the base station processor 80 and/or the accessory processor 86.

Preferably, the base station processor 80 communicates via the communication network 200 with the accessory processor 86 to facilitate transmission of the sensor data 100 (either the entire subset of data or a portion thereof) to the accessory processor 86 for storage in the accessory database 88.

In a preferred embodiment, the device subsystem 92 may include a hardware and/or software application that allows for the receipt of data 100 associated with the position of the probe 10 that has the capability to use the 802.11 protocol, Bluetooth communication and/or another linkage. For example, cellular communication and/or the communication network 200 may be used. Additional hardware and/or software applications may: (i) be enacted upon activating the device 10 within the work area 30; (ii) connect wirelessly to the base station processor 80 (e.g., via Bluetooth, WiFi and/or another linkage); and/or (iii) store the data 100 in the device database 72 for subsequent transmission to the base station database 82.

Device

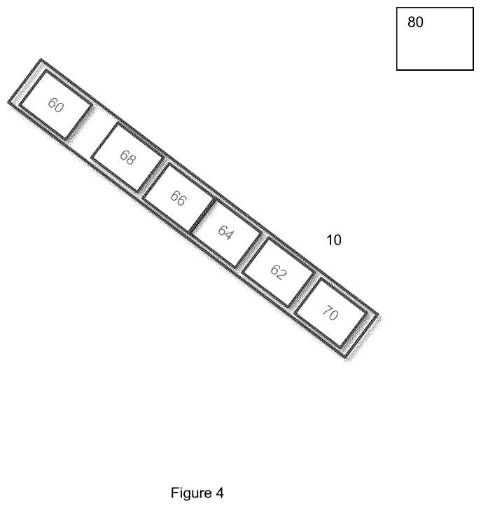

Referring now to FIG. 4, in preferred embodiments, the present invention includes a hand held probe 10 that may contain sensors including an accelerometer 64, a gyroscope 66 and/or magnetometers 60, 62. Each of the sensors 60, 62, 64, 66 may be tri-axial, to detect measurements along the x, y and/or z-axes. Each of the accelerometer 64, gyroscope 66 and/or magnetometers 60, 62 is preferably digital and communicates digital data 100 over a communication link (for example, communication network 200). The digital data 100 includes the sensor information (e.g., measurements along the x, y and/or z-axes). The probe 10 preferably has dimensions of a couple of centimeters in its longest dimension.

In preferred embodiments, the accelerometer 64 provides accelerometer data 100b including positional and/or angle (e.g., roll, pitch and/or yaw) information. In preferable embodiments, the positional and/or angle information is associated with the probe 10. The positional information may be from about 0.5 mm to about 2.5 mm relative accuracy over a distance of about 20 mm to about 300 mm and most preferably about 1mm relative accuracy. The accuracy may be relative to the former position of the probe 10. In some cases, the accelerometer 64 may have noise in the detected location, or location of interest, and acceleration information 100b (alternatively “acceleration data 100b”) and the accuracy may depend on the acceleration. The position information 102 is preferably the second integral of the acceleration provided by the acceleration data 100b. The accelerometer may also not generate angle perpendicular to the plane of gravity—i.e. yaw—providing only pitch and roll.

In preferred embodiments, the gyroscope 66 provides gyroscope data 100c including relative heading information. The accuracy of the relative heading information over 360 degrees may be to an accuracy of about 60 millidegrees relative to the sensor's (alternatively “probe's”) former position and most preferably about 0.06 degrees relative to the sensor's former position. In some cases, the gyroscope 66 may have noise in the detected angle information. The output 100c, or detected angle information 100c, may be integrated to determine the relative heading information from the angle movement information obtained from the gyroscope 66.

In preferred embodiments, the magnetometers 60, 62 provide magnetometer data 100a including both heading and position information relative to a magnetic field (not shown). Typically, the heading and position information is determined relative to the magnetic field of the Earth. The heading information may be determined with from about 360 degrees in yaw and most preferably with about 1 to about 0.1 degree accuracy. The position information may be determined with an accuracy of about +/−0.5 mm to about +/−2 mm. For the purposes of the present application, the yaw is preferably determined with the gyroscope 66. A typical magnetometer may be sensitive to perturbations in the magnetic field from local ferrous materials and electromagnetic interference and the magnetometer sensors 60,62 may be associated with low bandwidth, such that it may be slow for the sensor 60,62 to make a measurement and provide results. In addition, magnetometers require calibration before they can provide accurate results.

As shown in FIG. 4, preferable embodiments of the present invention include a probe 10 having up to two magnetometers 60, 62. The magnetic field vectors (not shown) detected by magnetometers 60, 62 may be subtracted to remove the magnetic field of the Earth. As may be understood by persons skilled in the relevant art, magnetic field strength decreases by the cube of the distance and is non-linear.

In preferable embodiments, the gyroscope 66 is adapted to provide a higher bandwidth and sense or detect changes in heading much quicker than magnetometers 60, 62. Magnetometers 60, 62 may have low bandwidth and may suffer from lag. The gyroscope data 100c may preferably be integrated to provide position information 102 which is used to provide rough (or estimated) course corrections and to provide a buffer for the magnetometers 60,62 to catch up (or determine changes in heading).

In preferable embodiments, the accelerometer 64 is adapted to correct for heading as accelerometers can typically sense, or detect, gravity very accurately and can measure the angle normal to gravity to within about 0.01°—as long as the accelerometers are not moving. In the case of moving accelerometers, gyroscopes may be used to remove, or mitigate, the effects of angular changes.

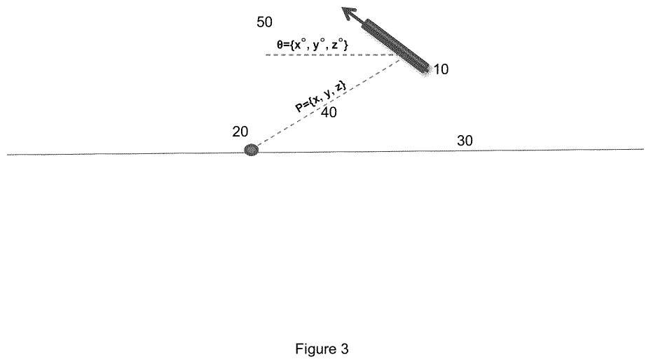

With reference to FIG. 3, in preferable embodiments, a second position sensor 20 (alternatively “reference magnet 20”) may be placed or positioned in the work area 30, proximate to the probe 10. The probe 10, including magnetometers 60,62 may detect the relative position 40 and angle 50 to the reference magnet 20. The reference magnet 20 may provide a magnetic field with a strength of between about 2 mT and about 4 mT and most preferably about 3 mT at a distance of about 50 mm. In preferable embodiments, the reference magnet 20 may be a button or bar magnet.

As best shown in FIG. 4, in preferable embodiments, a controller 68 receives digital sensor information 100 from the accelerometer 64, gyroscope 66 and magnetometers 60,62 and combines the data sources (or sensor information 100) into a position and heading information (or orientation) 102. The sensor data 100 preferably provides up to 9 degrees of freedom. Preferably, the sensor data 100 is time-stamped or associated with the time that the data 100 was detected, collected and/or recorded. Preferably, the position and heading information 102 may be transmitted from the controller 68 to the base station processor 80 and/or the accessory processor 86.

In preferable embodiments, as shown in FIG. 4, the probe 10 includes an accelerometer 64, a gyroscope 66 and a magnetometer 60, 62 and may be implemented with a single magnetometer. The physical location and orientation of the sensors 60, 62, 64, 66 within the probe 10 is preferably well defined so that the sensor data 100 from the sensors 60, 62, 64, 66 can be combined, analyzed and/or transmitted by the controller 68. The probe 10 may be moved in proximity to the reference magnet 20 (as depicted in FIG. 3).

In preferable embodiments, a processing engine, either running on the controller 68, or the base station processor 80 in communication with the probe 10 may use the sensor data 100 to determine the position and angle of the probe 10. The position and angle of the probe 10 may preferably be relative to the reference magnet 20.

In an embodiment, the instrument 70 associated with the probe 10 includes an ultrasound emitter/detector for obtaining ultrasound images of a human patient. The probe 10 may contain the accelerometer 64, gyroscope 66 and magnetometer 60, 62 as described above and the instrument 70 as depicted in FIG. 4. The instrument 70 may be integrated with the probe 10 in a single device or the probe 10 may be affixed, either permanently or removably, to the instrument 70 (not shown). In an embodiment, the probe 10 may be included in or with a sleeve (not shown) that is permanently or removably affixed to the instrument 70. Instrument data 104 may be communicated to the base station processor 80 and used in conjunction with the position and angle information 100c (e.g., generated by the gyroscope) of the probe 10. The specific orientation of the instrument 70 relative to the other sensors 60, 62, 64, 66 may be known based on (or predetermined from) the construction of the probe 10.

The reference magnet 20 may be placed on the body of a patient 15 to define the work area 30. For example, the reference magnet 20 may be incorporated into a stick pad (not shown) that is removably attached to the patient 15. The magnet 20 may be attached in a manner similar to ECG probes. In preferable embodiments, the magnet 20 is attached to the sternum of the patient so that the magnet 20 lies with a known orientation relative to the patient 15.

In other embodiments, the probe 10 may be used for other medical applications such as instruments used during surgery to determine the location and orientation of tools or diagnostic instruments associated with the probe 10. The probe 10 may be integrated with the tools or diagnostic instruments or may be affixed, either permanently or removably, to the tool or diagnostic instrument. Preferably, the probe 10 is used to track the position of surgical equipment, other sensors and transducers where accurate, repeatable measurements within a three-dimensional volume is desired. Preferably, the probe 10 of the present invention is adapted to track patient and/or anatomical movement to correct and/or maintain spatial integrity in a three-dimensional volume.

In preferable embodiments, the processors 68, 80 receive raw unprocessed sensor data 100 obtained from the sensors 60, 62, 64, 66 in real-time. A processing engine running on the processors 68, 80 preferably assigns a priority or weight to the data 100 received from each sensor 60, 62, 64, 66 based on the likely accuracy and/or bandwidth of a given sensor 60, 62, 64, 66. In this way, the sensor information 100 or data 100 may be transformed into a heading and position vector 102. The heading and position vector 102 may be updated regularly (e.g., at predetermined intervals), depending on the speed of the sensors 60, 62, 64, 66, the processing engine and the application.

In preferable embodiments, the processing engine application may operate on the probe 10 via the controller or embedded processor 68 (or “device processor 68”). The embedded processor 68 may operate at 16 bit or 32 bit. Alternatively, the processing engine application may operate at a base station processor 80 in communication with the probe 10 and the sensor data 100. The base station processor 80 may be a computer containing software to perform the processing of the sensor data 100. In an embodiment, the processing engine application may be remote from the base station processor 80, such as at an accessory processor 86 (e.g., a server or a cloud based server).

The probe 10 may preferably contain one or more interface features 74, such as buttons, switches, display screens, interactive screens, indicator lights/LEDs. The interface features 74 may allow the probe 10 to be turned on and off, perform configuration or setup functions, or interact with the base station processor 80. Interface features 74 may provide status, such as that the probe 10 is on and functioning properly, that there is an error that needs to be addressed, that some user action is required, or some other issue.

The probe 10 may contain or be affixed to additional instruments or sensors 70 such as for example a temperature sensor (not shown). The user may use the interface features 74 to activate or take a measurement using one or more of the additional sensors 70. The sensor data 104 from the additional sensors may be stored and/or communicated to the base station processor 80. The interface features 74, such as an LED may indicate to the user that a measurement using an additional sensor 70 should be taken, or has been captured successfully. Interface features 74 such as buttons, may activate the additional sensor 70 and cause the additional sensor 70 to send or store sensor data 104, such as the current temperature.

The probe 10 is preferably sealed to facilitate cleaning and sterilization, as with other medical instruments, so that it may be reused after use with other patients. Any interface features 74, such as on/off switches, or configuration buttons, are preferably also sealed with the body of the probe 10.

The probe 10 may preferably communicate wirelessly, such as using Bluetooth, WiFi, with the base station processor 80. Wireless communication may allow the probe 10 to be more easily manipulated during use since a cable is not required between the probe 10 and the base station 80.

The probe 10 may also preferably contain a power source 79a (e.g., a battery) for powering the sensors 60, 62, 64, 66, controller 68 and other electronics contained in the probe 10. The battery 79a is preferably rechargeable and may be recharged when the probe 10 is placed in or near a charging station (not shown). The charging station preferably uses wireless charging so that the probe 10 may remain sealed during charging and physical electrical connections are not required for use with the probe 10.

In an alternative, the probe 10 may have a wired connection with the base station processor 80. The wire connection may provide electrical power to the probe 10 to power the sensors 60, 62, 64, 66, controller 68 and other electronics housed in the probe 10. The wire connection may also provide a communications path between the probe 10 and the base station processor 80 to allow sensor information 100, and/or position and orientation information 102 to be communicated to the base station processor 80. If the probe 10 contains or is affixed to an instrument 70 (e.g., an ultrasound) the instrument control and sensor data 104 may also be communicated to the base station processor 80 on the wired connection.

Processors

Preferably, the processors 68, 80 are operatively encoded with one or more algorithms 801a, 801b, 802a, 802b, 803a, 803b, 804a, 804b, 805a, 805b (shown schematically in FIG. 2 as being stored in the memory 78a associated with the device subsystem 92 and/or the base station subsystem 94) which provide the processors 68, 80 with analysis logic 801a,b, data packet logic 802a,b, device status logic 803a,b, report generation logic 804a,b, and calibration logic 805a,b. Preferably, the algorithms 801a, 801b, 802a, 802b, 803a, 803b, 804a, 804b, 805a, 805b enable the processors 68, 80 to assess the sensor data 100 received from the controller 68 as well as any additional data that may be associated with the position of the probe 10 (e.g., instrument data 104). The base station processor 80 and/or the controller 68 are preferably operatively connected to one or more power sources 79a,b.

The base station processor 80 is preferably in communication with the device processor 68 and/or the accessory processor 86. Preferably, the base station processor 80 may be used to automatically: (i) collect the data associated with the probe 10 (e.g., sensor data 100, instrument data 104, calibration data 106); and (ii) combine and/or reconcile the data associated with the probe 10 (data 100, 104 and/or 106) and generate position and orientation data 102.

In accordance with the present invention, analysis includes, for example, combination, integration, etc. of magnetometer data 100a, accelerometer data 100b, gyroscope data 100c, instrument data 104 and/or calibration data 106 to facilitate the generation of position and orientation data 102. In some preferable embodiments, if all of the collected data 100, 104, 106 is not required to determine the position and orientation data 102, then the analysis will only include the required collected data 100, 104, 106 to determine the position and orientation data 102.

Preferably, the device processor 68 and/or the base station processor 80 automatically determine, at regular intervals (e.g., determined by the user), the position and orientation data 102. Some of the position and orientation data 102 may include the status of the probe 10 and of any communication link between the device processor 68, the base station processor 80 and the accessory processor 86.

Data Packets

Preferably, the data 102, 104, 106 are divided or disassembled into a plurality of manageable and discrete data packets prior to transmission by the processors 68, 80 and 86 using the data packet algorithm 802a,b. Following transmission, the plurality of discrete data packets are preferably automatically joined or reassembled into the corresponding data 100, 104, 106 by the processors 68, 80, 86 using the data packet algorithm 802a,b.

The data packets may be data packets in the conventional sense, or they may be more akin to data “chunks”. That is, the present invention contemplates the use of any suitable way of segmenting and transmitting the data 100, 104. 106 for subsequent re-assembly. For example, all data associated with the position of the probe 10 may be transmitted together. Any positions of the probe 10 for which only a partial record is received, or for which no data and/or corrupted data is received may be flagged for correction, follow-up and/or replacement. It is implicit from all the foregoing that, when appropriate, data packets in the conventional sense may be suitable for incorporation in and/or use with the present invention.

Communication Interruption

Preferably, if the transmission of the data 100, 104, 106 from the device processor 68 is terminated, severed, interrupted and/or impaired—whether to the base station processor 80 and/or the accessory processor 86—then the transmitted data 100, 104, 106 that has been received by the base station database 82 and/or the base station processor 80 may be deleted (from the device subsystem 92). Un-transmitted data 100, 104, 106 that has not been received by the base station database 82 and/or the base station processor 80 may be received by and maintained on the device database 72 for subsequent transfer to the base station database 82 and/or the base station processor 80 when communication is restored.

Presentation

The processors 68, 80 preferably generate a signal for presentation of the position and orientation data 102 in the form of an image or text to the user and/or a third party (e.g., an administrator) of the system 90. The data 102 may be presented by the system 90 using a graphical user interface associated with the device processor 68 and/or the base station processor 80. As shown in FIG. 1, the data 102 may be presented using one or more reports 110.

FIG. 2 schematically illustrates, among other things, various input/output devices 84 (including the GUI 84a, a printer 84b, speakers 84c, and LED 84d) associated with the base station database 88, the device subsystem 92 and/or the base station subsystem 94.

The GUI 84a may include a touchscreen, a display with or without a “point-and-click” mouse or other input device. The GUI 84a enables (selective or automatic) display of the data 100, 102, 104, 106 determined by the processors 68, 80—whether received directly therefrom and/or retrieved from the databases 72, 82, 88.

In preferable embodiments, the probe 10 of the present invention includes a light pipe (not shown). The light pipe uses LEDs in conjunction with a light guide to illuminate the housing of the probe 10 from the inside to provide an indication of the state of the various sensors 60, 62, 64, 66, including normal functions.

Preferably, the system 90 includes two sensors, the first position sensor 10 that is associated with the instrument 70 (e.g., ultrasound transducer) and the second position sensor 20 (e.g., reference magnet) that is associated with the patient. The first position sensor 10 is preferably mounted on a sleeve that is configured for a specific make and/or model of the instrument (e.g., an ultrasound transducer). That second position sensor is associated or attached to the patient using, for example, medical grade double-sided tape.

The system 90 includes a report generation unit for generating the reports 110. Among others, the following reports 110 may be generated, based upon the data 100, 102, 104: activity reports; status reports; probe position reports; user customized reports; and/or communication reports.

Method

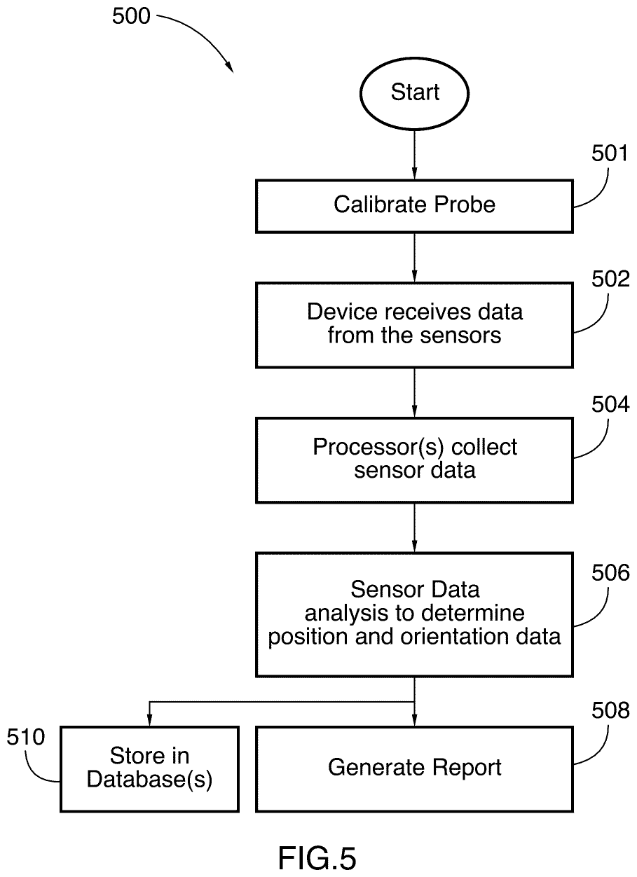

FIG. 5 depicts steps of a method 500 to determine position and orientation data 102 using the sensor data 100. Persons skilled in the art will understand that the accelerometer data 100b, the gyroscope data 100c, and/or the magnetometer data 100a may be used in the method 500. Method 500 is suitable for use with the system 90 and device 10 described above and shown in FIG. 1, but is not so limited.

As shown in FIG. 5, the method 500 includes the following steps, among others: a start step; a probe calibration step 501; a sensor data reception step 502; a sensor data collection step 504; a position and orientation data generation step 506; a report generation step 508; and/or a step 510 of storing the position and orientation data in the databases 82, 86.

Preferably, the probe calibration step 501 includes calibration of the different sensors 60, 62, 64, 66 separately using different procedures. The accelerometer 64 is preferably calibrated using a gimbal that is rotated through roll, pitch and/or yaw for the full 360 degrees. The processor 68 collects and assembles the data 100 into a three-dimensional sphere of information. This calibration is preferably conducted for each probe 10. The gyroscope 66 is preferably positioned on a stationary surface and the DC bias is recorded for later use by the processor 68. A simple subtraction of the DC bias is all that is required for calibration. The magnetometer 60, 62 is preferably calibrated once the fully assembled sensor 10 is mounted to the instrument sleeve. Preferably, the first step is completed during installation whereby a full sphere calibration is performed by moving (i.e., rotating) the sensor 10. The second step is preferably completed when the sensor 10 is paired with the second position sensor 20 for alignment prior to clinical examination. Preferably, the accelerometer 64 and gyroscope 66 are calibrated during manufacturing of the probe 10. The reference magnet 20 may preferably be calibrated by moving it around a specific movement with the use of a robot, for example, to characterize and fit the magnetic field to a predetermined magnetic field equation. Calibration data 106 includes calibration information associated with the calibration of the accelerometer 64, the gyroscope 66 and the magnetometers 60, 62. In an alternate embodiment, calibration information associated with the calibration of the accelerometer 64, the gyroscope 66 and the magnetometers 60, 62 may be included as sensor data 100.

It will be appreciated that, according to the method 500, the sensor data 100 is collected by the probe 10. One or more components 300 of the device 10 may collect information that is preferably recorded as sensor data 100 and/or instrument data 104 in the device database 72. The processors 68, 80 are used to automatically: collect the data 100, 104, 106; analyze the data 100, 104, 106 to generate position and orientation data 102; and generate a report 110 which includes the collected 100, 104, 106 and/or analyzed data 102 preferably presented to the user (or a third party). Thus, according to the invention, the method 500 operatively facilitates the analysis of, for example, sensor data 100 to determine the position and orientation of the probe 10.

The computer readable medium 78, shown in FIG. 2, stores executable instructions which, upon execution, analyzes sensor data 100, instrument data 104 and/or calibration data 106. The executable instructions include processor instructions 801a, 801b, 802a, 802b, 803a, 803b, 804a, 804b, 805a, 805b for the processors 68, 80 to, according to the invention, perform the aforesaid method 500 and perform steps and provide functionality as otherwise described above and elsewhere herein. The processors 68, 80 encoded by the computer readable medium 78 are such as to receive data 100, 104, 106 perform an analysis (e.g., integration, combination, etc.) on the data 100, 104, 106 to determine position and orientation data 102, generate a report 110 based on the analysis, and transmit the data 100, 102, 104, 106 to the device database 72, base station database 82, and/or the accessory database 88. Thus, according to the invention, the computer readable medium 78 facilitates the use of the processors 68, 80 to operatively facilitate the analysis of the data 100, 104, 106 of the probe 10.

Thus, the system 90, method 500, device 10, and computer readable medium 78 operatively facilitate the determination of the position and orientation of an instrument 70 associated with the probe 10.

Data Store

A preferred embodiment of the present invention provides a system 90 including data storage 72, 82, 88 that may be used to store all necessary data 100, 102, 104, 106 required for the operation of the system 90. A person skilled in the relevant art may understand that a “data store” refers to a repository for temporarily or persistently storing and managing collections of data 100, 102, 104, 106 which include not just repositories like databases (a series of bytes that may be managed by a database management system (DBMS)), but also simpler store types such as simple files, emails, etc. A data store in accordance with the present invention may be one or more databases, co-located or distributed geographically. The data being stored may be in any format that may be applicable to the data itself, but may also be in a format that also encapsulates the data quality.

The foregoing description has been presented for the purpose of illustration and maybe not intended to be exhaustive or to limit the invention to the precise form disclosed. Other modifications, variations and alterations are possible in light of the above teaching and may be apparent to those skilled in the art, and may be used in the design and manufacture of other embodiments according to the present invention without departing from the spirit and scope of the invention. It may be intended the scope of the invention be limited not by this description but only by the claims forming a part of this application and/or any patent issuing herefrom.

1. A position probe system for use with an ultrasound transducer by a user with a patient, wherein the system comprises:
(a) a first position sensor comprising: (i) an accelerometer adapted to receive accelerometer data associated with the first position sensor; (ii) a gyroscope adapted to receive gyroscope data associated with the first position sensor; and (iii) a magnetometer adapted to receive magnetometer data associated with the first position sensor;
(b) one or more processors operative to: (i) electronically receive the accelerometer data, the gyroscope data and the magnetometer data; and (ii) analyze the accelerometer data, the gyroscope data and the magnetometer data using an analysis algorithm to automatically generate position and orientation data associated with the first position sensor; and
(c) one or more databases to electronically store the accelerometer data, the gyroscope data, the magnetometer data and the position and orientation data;
wherein the system is operative to facilitate the determination of the position and orientation of the first position sensor.

2. The position probe system of claim 1, further comprising a second position sensor associated with the patient to facilitate the determination of the position and orientation of the first position sensor relative to the patient.

3. The position probe system of claim 2, wherein the second position sensor is a reference magnet.

4. The position probe system of claim 2, wherein the second position sensor is secured to the patient to compensate for movement of the patient during the determination of the position and orientation of the first position sensor.

5. The position probe system of claim 1, wherein the accelerometer data, the gyroscope data, and the magnetometer data comprise an x-axis, a y-axis, and/or a z-axis.

6. The position probe system of claim 1, wherein the accelerometer data, the gyroscope data, and the magnetometer data is time-stamped.

7. The position probe system of claim 1, wherein the first position sensor is associated with an ultrasound transducer.

8. The position probe system of claim 7, wherein the first position sensor is removably mounted to the ultrasound transducer.

9. The position probe system of claim 7, wherein the first position sensor is integral with the ultrasound transducer.

10. The position probe system of claim 1, wherein the one or more databases comprises a device database, local to the first position sensor, to electronically store the accelerometer data, the gyroscope data, the magnetometer data and the position and orientation data.

11. The position probe system of claim 1, wherein the first position sensor further comprises interface features to facilitate interaction by the user with the sensor.

12. The position probe system of claim 1, wherein the first position sensor further comprises a light pipe to illuminate the sensor.

13. (canceled)

14. (canceled)

15. (canceled)

16. (canceled)

17. (canceled)

18. (canceled)

19. (canceled)

20. (canceled)

21. (canceled)

22. (canceled)

23. A first position sensor, operated by a user, for use with an ultrasound transducer wherein the first position sensor comprises:
(a) an accelerometer adapted to collect accelerometer data associated with the first position sensor;
(b) a gyroscope adapted to collect gyroscope data associated with the first position sensor;
(c) a first magnetometer adapted to collect magnetometer data associated with the first position sensor;
(d) one or more processors operative to receive the accelerometer data, the gyroscope data and the magnetometer data and automatically apply an analysis algorithm to generate position and orientation data associated with the first position sensor, with the accelerometer data, the gyroscope data and the magnetometer data electronically stored in one or more databases;
whereby, the first position sensor is operative to facilitate determination of the position and orientation of the first position sensor.

24. The first position sensor of claim 23, further comprising a second magnetometer adapted to collect the magnetometer data associated with the first position sensor.

25. The first position sensor of claim 23, wherein the accelerometer, the gyroscope, the first magnetometer and the second magnetometer are tri-axial.

26. The first position sensor of claim 23, wherein the one or more databases comprises a device database local to the first position sensor to electronically store the accelerometer data, the gyroscope data and the magnetometer data.

27. The first position sensor of claim 23, further comprising one or more interface features to facilitate interaction between the user and the sensor, wherein the one or more interface features comprise buttons, switches, display screens, interactive screens, and/or indicator lights.

28. (canceled)

29. The first position sensor of claim 23, further comprising a light pipe adapted to illuminate the sensor.

30. The first position sensor of claim 23, wherein the first position sensor processor is in communication with and/or detects a second position sensor associated with a patient to facilitate the determination of the position and orientation of the first position sensor relative to the patient.

31. (canceled)

32. A position probe system for use with an ultrasound transducer by a user with a patient, wherein the system comprises:
(a) a first position sensor comprising: (i) an accelerometer adapted to receive accelerometer data associated with the first position sensor; and (ii) a gyroscope adapted to receive gyroscope data associated with the first position sensor;
(b) one or more processors operative to: (i) electronically receive the accelerometer data and the gyroscope data; and (ii) analyze the accelerometer data and the gyroscope data using an analysis algorithm to automatically generate position and orientation data associated with the first position sensor; and
(c) one or more databases to electronically store the accelerometer data, the gyroscope data, and the position and orientation data;
wherein the system is operative to facilitate the determination of the position and orientation of the first position sensor
